(19) United States
(12) Patent Application Publication
(10) Pub. No.: US 2009/0023189 A1
(43) Pub. Date: Jan. 22, 2009

(54) APPARATUS AND METHODS FOR PREPARATION OF SUBTANTIALLY UNIFORM EMULSIONS CONTAINING A PARTICLE

(75) Inventors: Aldrich N.K. Lau, Palo Alto, CA (US); Jonathan M. Cassel, Half Moon Bay, CA (US); John A. Bridgham, Hillsborough, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 12/119,475

(22) Filed: May 12, 2008

Related U.S. Application Data
(60) Provisional application No. 60/924,544, filed on May 18, 2007.
(60) Provisional application No. 60/924,664, filed on May 24, 2007.

(51) Int. Cl. C12P 19/34; C12M 1/40

(52) U.S. Cl. 435/91.2; 435/289.1

(57) ABSTRACT

Methods and systems for forming water-in-oil emulsions are described. For example, an apparatus is described which includes: a first compartment containing a plurality of particles dispersed in an aqueous phase; a second compartment containing an oil phase; a porous layer separating the first and second compartments; and a device for applying pressure to the first compartment. A method is described which includes: moving an oil phase relative to a surface of a porous layer while simultaneously forcing an aqueous composition comprising particles through the porous layer and into the flowing dispersion medium thereby forming droplets of the aqueous composition containing particles dispersed in the oil phase. The aqueous composition can include one or more nucleic acid templates and reagents for amplifying the nucleic acids such as PCR reagents. A porous partition is described comprising a first and second major surfaces and at least two straight through pores comprising a cross sectional shape selected from a polygon, an oval, an oblong, a dumbbell, a bowtie and irregular shapes thereof. Aqueous droplets containing an oligonucleotide attached to a particle and reagents can be used as a microreactor for nucleic acid amplification.

This application claims benefit of priority to U.S. Provisional Application Ser. Nos. 60/924,544 filed May 18, 2007 and 60/924,664 filed May 24, 2007, each of which is incorporated by reference in their entirety.

The section headings used herein are for organizational purposes only and should not be construed as limiting the subject matter described herein in any way.

### FIELD

This application relates generally to systems and methods for forming substantially uniform water-in oil emulsions comprising a continuous oil phase and discrete droplets of an aqueous phase containing an oligonucleotide attached to a particle.

### INTRODUCTION

Emulsions are utilized in food preparation, chemical, cosmetic, and pharmaceutical processes in which two immiscible liquids are combined. Emulsions having uniformly sized emulsion droplets, also termed monodispersed emulsion, are more stable than polydispersed emulsions. A particle contained within the emulsion droplet can provide additional properties to an emulsion.

An emulsion can be prepared by vortexing and stirring two immiscible liquids or by forcing a first immiscible phase through pores in a membrane and into a second immiscible, continuous phase. In either process there is variability in the size of the emulsion droplets and the stability of the emulsion. Numerous procedures have been developed to improve the uniformity and stability of the emulsion.

Tadao Nakashima and coworkers were the first to report the use of a Shirasu Porous Glass (SPG) membrane for preparing emulsions (Ceram. Jpn. 1986, 21, 408; U.S. Pat. No. 4,657,875 (1987)). In order to prepare an inverse emulsion (water-in-oil) by passing the aqueous phase through the pores of a membrane, at least the membrane surface facing the continuous oil phase has to be hydrophobic (C.-J. Cheng, et al., J. Colloid Interface Sci. 2006, 300:375-382; N. Yamazaki, et al., J. Dispersion Sci. & Tech. 2003, 24:249-257; K. Suzuki, Reza Kenkyu 1993, 21:26-31.) The hydrophobicity of the membrane and interior pore surfaces has an impact on emulsion size, stability and rate of formation. Methods for altering the surface properties of the membrane and the interior surface of the pores of the membrane can be widely divergent.

Nanomi Emulsification Systems (Enschede, Netherlands) prepares track-etched porous membranes using a silicon wafer for the membrane (J. Wissink, et al., US 20070227591A1). Pores with ≧2μ diameter, comprising auxiliary structures along its longitudinal axis, are formed in the silicon wafer by reactive ion etching or other lithographic techniques. This is followed by subjecting the membrane to chemical vapor deposition (CVD) with a silane reagent to render all membrane surfaces hydrophobic, including the interior surface of the pores. Hydrophobicity enables the aqueous phase to form a droplet on the membrane surface, instead of a puddle, as it is forced to pass through the pore to the other side of the membrane. Since the critical pressure Pc is proportional to the contact angle, cos θ, of the discrete phase on the membrane, relatively high pressure is necessary to force the aqueous phase through the pores and only a small portion of the pores, if the interior walls are hydrophobic (C. Charcosset, et al., J. Chem. Tech. Biotech. 2004, 79, 209-218), allow the aqueous phase to break through the membrane resulting in differing rates of formation and variability in emulsion droplet size.

### SUMMARY

In accordance with the embodiments, there is disclosed a device for forming a plurality of substantially uniform-size emulsion droplets, at least one emulsion droplet comprising a particle including: a first chamber containing an aqueous phase including a plurality of particles; a second chamber containing an oil phase which includes a water-immiscible liquid; and a partition separating the first chamber from the second chamber, said partition comprising at least a first major surface, a second major surface opposite the first major surface, and at least two defined straight through pores connecting the first major surface to the second major surface, where said first major surface forms a wall of the first chamber and said second major surface forms a wall of the second chamber; wherein when said aqueous phase passes through two of said at least two straight through pores to said oil phase said aqueous phase forms a plurality of discrete substantially uniform-size emulsion droplets, at least one of the plurality of droplets includes at least one particle of the plurality of particles, said at least one particle comprising at least one nucleic acid attached to the particle.

In another embodiment, there is also disclosed a partition through which to pass a first phase into a second phase to form substantially uniform-size emulsion droplets, including: a first major surface; a second major surface opposite the first major surface; and at least two straight through pores including a cross sectional shape selected from the group consisting of a polygon, an oval, an oblong, a dumbbell, a bowtie and irregular shapes thereof a and an interior wall traversing the partition between the first major surface and the second major surface, and wherein said partition is adapted to form substantially uniform-size emulsion droplets including the first phase in the second phase.

In yet another embodiment, there is disclosed a method of forming substantially uniform-size emulsion droplets including: forcing an aqueous phase including a plurality of particles in contact with the first major surface of a partition through at least two straight through pores in the partition and into a dispersion medium, and simultaneously, moving the dispersion medium parallel to and in contact with a second major surface of the partition wherein the second major surface is opposite the first major surface, thereby forming substantially uniform-size emulsion droplets of the aqueous phase dispersed in the dispersion medium, and wherein a plurality of the droplets include at least one particle.

In the following description, certain aspects and embodiments will become evident. It should be understood that a given embodiment need not have all aspects and features described herein. It should be understood that these aspects and embodiments are merely exemplary and explanatory and are not restrictive of the invention.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

There still exists a need for improved systems and methods for forming uniformly sized droplets of an aqueous phase containing at least one particle, dispersed in an oil phase, an inverse emulsion. Therefore, it is desirable that the surface of the membrane in contact with the continuous oil phase is hydrophobic while the membrane's interior wall surfaces are hydrophilic which results in lowering the applied pressure necessary to force the aqueous phase through the porous membrane partition for the preparation of inverse emulsion droplets. Methods which prepare the porous membrane surface facing the aqueous phase and interior wall surfaces to have hydrophilic characteristics and the membrane surface facing the oil phase to have hydrophobic characteristics will improve the formation of substantially uniform inverse emulsion droplets. Inverse emulsion droplets containing particles could be used as micro reactors for conducting nucleic acid amplifications such as polymerase chain reaction (PCR) amplification.

These and other features of the present teachings are set forth herein.

### BRIEF DESCRIPTION OF THE DRAWING

The skilled artisan will understand that the drawings described below are for illustration purposes only. The drawings are not intended to limit the scope of the present teachings in any way.

FIG. 1 shows a perspective view of a device for forming emulsions.

FIG. 2 shows a cross sectional view of a device for forming emulsions.

FIG. 3 shows an expanded portion of the cross sectional view of FIG. 2.

FIG. 4A-4F show examples of various straight through pore shapes and arrangements of the pores within the partition.

FIG. 5 shows the cross sectional shapes of exemplary pores.

FIG. 6 shows a cross sectional view of surface treatments for the partition of FIG. 3.

FIG. 7 shows an expanded portion of the cross sectional view of FIG. 6.

FIG. 8 represents a block diagram showing the general method of forming the substantially uniform-size inverse emulsion.

### DETAILED DESCRIPTION

For the purposes of interpreting of this specification, the following definitions will apply and whenever appropriate, terms used in the singular will also include the plural and vice versa In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the phrase “membrane,” “partition,” “layer,” and “film” are interchangeable and not intended to be limiting.

As used herein, the phrase “nucleic acid,” “oligonucleotide”, and polynucleotide(s)” are interchangeable and not intended to be limiting.

As used herein, “discrete aqueous phase”, “aqueous phase” and “oil immiscible liquid” are interchangeable and not intended to be limiting.

As used herein, the phrase, “aqueous phase” refers to an oil immiscible liquid.

As used herein, the phrase, “oil phase” refers to a water immiscible liquid. As used herein, “continuous phase”, “continuous oil phase”, “oil phase”, “water immiscible liquid”, and dispersion medium are interchangeable and not intended to be limiting.

As used herein, the phrase, “discrete phase” refers to the emulsified aqueous phase within an oil phase.

As used herein, the phrases, “dispersed phase” and “disperse phase” refer to an emulsified phase within an immiscible liquid. To illustrate, in a normal emulsion the oil phase is emulsified into the aqueous phase and can be said to be “dispersed” in the aqueous phase. Conversely, in an inverse emulsion, the aqueous phase is emulsified into the oil phase and can be said to be “dispersed” in the oil phase.

As used herein, the phrase “through pore” refers to a pore which connects a first major surface to a second major surface of a porous layer.

As used herein, the phrase “straight through pore” refers to a pore which connects a first major surface to a second major surface of a porous layer and through which a straight line can be drawn that does not touch or intersect the wall of the pore. “Straight through” pores are also referred to as “track etched” pores.

As used herein, the phrase “longitudinal axis” refers to the straight line drawn through a straight through pore and which does not touch or intersect the interior wall of the straight through pore.

As used herein, the phrase “interior wall” refers to the surface of the straight through pore within the porous membrane which connects a first major surface to a second major surface of a porous layer.

As used herein, the phrase, “substantially uniform” refers to the size and volume of an emulsion droplet formed by the device and methods disclosed herein. The plurality of emulsion droplets formed having a percent coefficient of variation of at least 5% to 20% and between at least 10% to 15% in size and volume.

The systems and methods described herein are equally adaptable to either a normal i.e., oil-in-water emulsion or an inverse i.e., water-in-oil emulsion. The method disclosed for forming the emulsion droplet containing an oligonucleotide attached to a particle includes forcing an aqueous phase containing a particle with an oligonucleotide attached through a partition having straight through pores separating two immiscible phases.

The emulsion droplets formed by the membrane emulsion device can be monodispersed, substantially uniform droplets in size and shape. One of skill in the art will appreciate the modifications and treatments to the membrane surface. interior walls and the pore shape and the two immiscible phases appropriate to obtain the desired emulsion.

Systems and methods for the preparation of inverse (i.e., water-in-oil) emulsions comprising particles entrapped in aqueous droplets are described herein. The particle containing aqueous droplets can be used for performing nucleic acid amplification (e.g., polymerase chain reaction) processes.

Reference will now be made in detail to several exemplary aspects of the disclosure, which are illustrated in the accompanying drawings. Whenever possible, the same reference numbers will be used throughout the drawings to refer to the same or like parts.

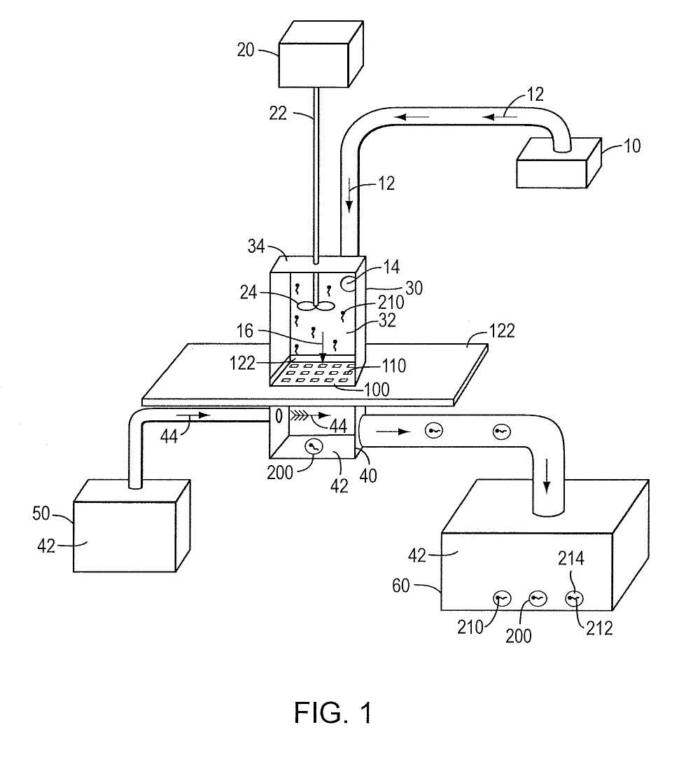

FIG. 1 schematically illustrates an exemplary device for use in the formation of monodispersed, substantially uniform-size emulsions and inverse emulsions, in accordance with various exemplary aspects of the present disclosure.

The apparatus may comprise at least one compartment 40 containing a continuous oil phase 42. The oil phase 42 may also comprise a surfactant. The surfactant can be an ionic or a non-ionic surfactant. This compartment 40 can be separated by a porous partition 100, which comprises straight through pores 114, from at least one compartment 30 containing the aqueous phase 32, a first composition in which at least a plurality of particles 210 are suspended.

The apparatus may include a device 10 for pressurizing the contents of the compartment 30 containing the aqueous phase 32. This device 10 may comprise a tank containing pressurized gas which is connected to the compartment 30 at an opening 14 containing the aqueous phase 32. The device may also comprise a wall 34 which is movable relative to the porous partition 100 and which can be moved to reduce the volume of the compartment 30 containing the aqueous phase 32. Other devices for pressurizing the contents of the compartment containing the aqueous phase 32 could also be used.

The apparatus may also include a device for moving the aqueous phase 32 to provide mechanical stirring of the aqueous phase 32. A mechanical stirrer can include a motor 20 having a shaft 22 between the motor 20 and leading to a blade 24 positioned within the compartment 30 containing the aqueous phase 32.

The apparatus may also comprise a device 50 for moving the continuous oil phase 42 relative to the surface 104 of the porous partition 100. This device 50 can comprise a pump adapted to pump the continuous oil phase 42 over the surface 104 of the porous partition 100. The flow of the oil phase 42 can be a laminar flow 44 which is parallel to the second major surface 104 of the porous partition 100 and perpendicular to the flow 16 of the aqueous phase 32 through the straight through pores 114 of the porous partition 100. The device may also comprise a stirring device or an agitation device adapted to stir or agitate the continuous oil phase 42 during emulsification. The agitation device can be a mechanical device, including, but not limited to, a vortexing device, a rocker, a shaker, an orbit shaker, or a sonicator. This list is not intended to be limiting and other devices for flowing or otherwise moving the continuous phase over the surface of the porous partition during emulsification could also be used.

The apparatus may also comprise a reservoir 60 for collecting the substantially uniform-size emulsion droplets 200 which are carried from the compartment 40 by the continuous oil phase 42 to the reservoir 60.

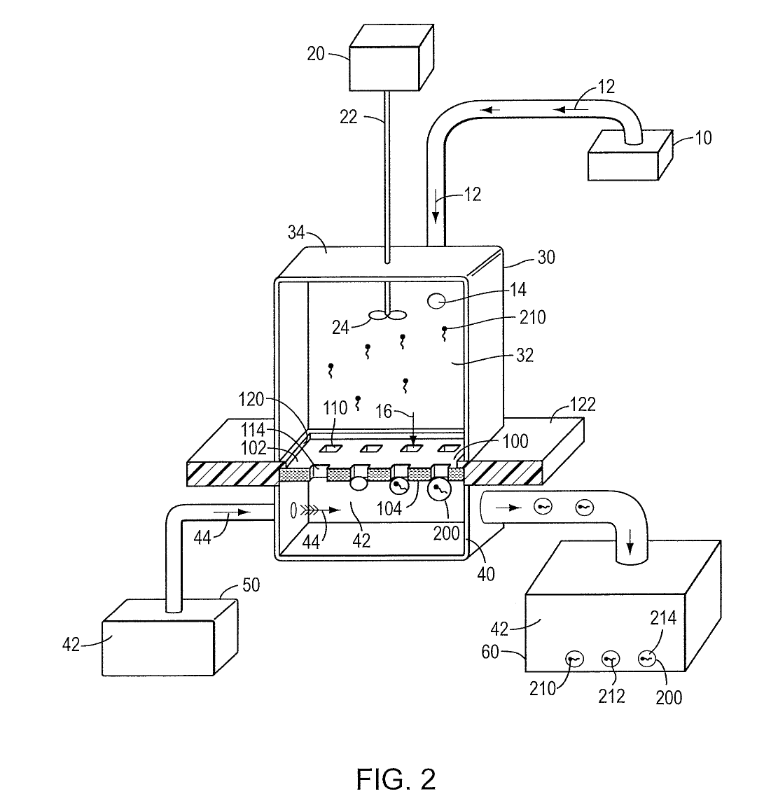

FIG. 2 schematically illustrates the cross section of the exemplary device of FIG. 1 for use in the formation of monodispersed, substantially uniform-size inverse emulsion droplets 200, in accordance with various exemplary aspects of the present disclosure. Although FIG. 2 illustrates the formation of inverse emulsions, one of skill in the art can envision modifications to the device of FIGS. 1 and 2 to form normal emulsions.

The apparatus can comprise at least one compartment 40 containing a continuous oil phase 42. The oil phase 42 can also comprise a surfactant. The surfactant can be an ionic or a non-ionic surfactant. This compartment 40 can be separated by a porous partition 100, which comprises straight through pores 114, from at least one compartment 30 containing the aqueous phase 32 in which a plurality of particles 210 are suspended. Attached to at least some of the plurality of particles 210 is at least one oligonucleotide 212.

The continuous phase 42 comprises a water immiscible liquid (e.g., an oil phase). Exemplary water immiscible liquids include, but are not limited to, silicone oils (including, but not limited, to poly(dimethylsiloxane), poly(methylphenylsiloxane), and their copolymers), petroleum special (Fluka), a saturated, or unsaturated aliphatic hydrocarbon, its halogenated derivatives, and combination thereof. The aliphatic hydrocarbons can be normal or branched, for example, but not limited to, hexane, isooctane, decane, dodecane, 1-dodecene, pentadecane, hexadecane, petroleum ethers, and mineral oils), heptadecane (bp 302° C.), heptamethylnonane (bp 240° C.), heptadecene (bp 159° C./11 mm Hg), perfluorotridecane (bp 196° C.) and FLUORINERT™ Electronic Liquid FC-770 (obtained from 3M, St. Paul, Minn.), aromatic hydrocarbons (including, but not limited to, benzene, toluene, cumene, alkylbenzenes and alkylarylbenzenes), esters (including but not limited to 1,4-dioctyl phthalate), fluorinated hydrocarbons (including, but not limited to, FLUORINERT™ FC-75 (3M) and CTSOLV-100 (Asahi Glass) and other halogenated hydrocarbons, and perfluoropolyethers, including, but not limited to FOMBLIN®, (d 1.88-1.92 g/cm³, viscosity 60-1500 cSt, obtained from Ausimont USA, Inc. (Thorofare, N.J.) and DEMNUM™ (Daikin Industries, Japan). Additional water immiscible liquids that can be used include, but are not limited to, naturally occurring oils such as vegetable oils (i.e., saturated and unsaturated fatty acids and derivatives).

Those having ordinary skill in the art can appreciate that any water immiscible liquid with a boiling temperature (bp) above 96° C. can be used as a continuous phase 42. The continuous phase liquid can be an aromatic hydrocarbon, its derivative and combination thereof, for example, but not limited to, 2,3,4,5,6-pentafluoroanisol (bp 139° C.), 1,3,5-trimethylbenzene (bp 166° C.), hexylbenzene (bp 226° C.). The continuous liquid phase can be an ester, for example, but not limited to, dioctyl terephthalate (bp 400° C.) and diisobutyl phthalate (bp 327° C.). The continuous liquid phase can have densities ranging from 0.5 to 3.0 g/cm³, for example, but not limited to, hexane (d 0.66 g/cm³, bp 69° C.), bromobenzene (d 1.49 g/cm³, bp 159° C.), perfluorokerosene (d 1.94 g/cm³, bp 210-240° C.), and tetrabromoethane (d 2.97 g/cm³, bp 190° C.) and can have a viscosity ranging from 0.5 to 100 mPa·s, for example, but not limited to, trichloroethylene (0.545 mPa·s, bp 87° C.), pentachlorethnae (2.254 mPa·s, bp 162° C.), hexadecane (3.03 mPa·s, bp 287° C.), dimethyl phthalate (14.4 mPa·s, bp 282° C.), and heavy mineral oils (˜70 mPa·s).

The continuous phase 42 can also contain an ionic or a non-ionic surfactant. Exemplary non-ionic surfactants that can be used in the continuous phase include, but are not limited to, the SPAN™ series surfactants, for example SPAN™ 80, the BRIL™ series surfactants, for example BRIL™ 72, the TETRONIC™ series surfactants, for example, TETRONIC™ 901, polyethylene glycol (2 e.o.) monostearate (Wako, Japan), and ABIL® EM-90 (Degussa). This exemplary list is not intended to be limiting and other ionic and non-ionic surfactants including other surfactants comprising fluorinated moieties can also be used. The hydrophilic-lipophilic balance (HLB) value of the ionic or non-ionic surfactant can be from 1 to 10, from 2 to 6, from 3 to 5 and so on.

The aqueous phase 32 can be an aqueous solution comprising reagents (e.g. reagents for amplifying nucleic acids). Exemplary reagents include, but are not limited to, magnesium chloride and biomolecules such as deoxynucleoside triphosphates (dNTP's), enzymes, beads or particles bearing covalently or non-covalently attached oligonucleotides, template, buffers, and other additives which are useful for enhancing polymerase chain reaction (PCR) efficiency and/or specificity. The aqueous phase 32 can also contain an ionic or a non-ionic surfactant. Non-ionic surfactants include, but not limited to, the TWEEN™ series surfactants, for example TWEEN™ 20, the PLURONIC® series surfactants (amphiphilic block copolymers), for example PLURONIC® F38, the SPAN™ series of surfactants, for example SPAN™ 20, amphiphilic diblock copolymers, including, but not limited to, poly(dimethylsiloxane-block-ethylene oxide), poly(methylphenylsiloxane-block-ethylene oxide), poly(dimethylsiloxane-block-2-hydroxyethylacrylate), and poly(alkylene-block-2-hydroxyethyl acrylate). The ionic or non-ionic surfactant in the aqueous phase 32 can have a hydrophilic-lipophilic balance (HLB) value ranging from 5 to 40, from 10 to 40, from 20 to 40, from 5 to 10, from 10 to 20, and so on.

The aqueous phase 32 can comprise a water-soluble polymer. The water soluble polymer can be added to the aqueous phase 32 to adjust the density and/or the viscosity of the aqueous phase 32 in order to control the effectiveness of emulsification and/or to improve emulsion stability. For example, a water-soluble bromine- or chlorine-substituted polymer can be added to the aqueous phase 32 to increase the density of the aqueous phase. A water-soluble polymer with a relatively high molecular weight, e.g., 1 million to 10 million MW, can be added to increase the viscosity of the aqueous phase 32. Water soluble polymers that can be added to the aqueous phase include, but are not limited to, water-soluble polyacrylamides, water-soluble poly(N,N-dimethylacrylamide), water-soluble poly(ethylene glycols), water soluble poly(ethylene oxides), their derivatives and combinations thereof.

The device can comprise particles 210 which can be a solid material that is insoluble in both the aqueous phase 32 and continuous phase 42. As illustrated in FIGS. 1 and 2, the aqueous phase 32 includes particles 210 which pass through the membrane 100 and are incorporated into or within the resulting emulsion droplets 200. The particles can comprise, for example, a material such as, but not limited to, metal, metal oxide, metal halide, metal hydroxide, silicon, silicon dioxide, silica, quartz, glass, glassy carbon, carbon, polymer, or blends and combinations thereof. The particles can have an irregular shape or a regular shape such as a cylinder, a sphere, or a disk. The size of the particle can range from 0.1 to 100 microns. The surface of the particle can be physically or chemically modified. For example, the surface of the particles can be modified to comprise immobilized polynucleotides 212. The immobilized polynucleotides can serve, for example, as PCR primers in emulsion PCR (ePCR) reactions. The surface of the particles can also be modified to contain other reactive groups for subsequent reactions such as, for example, bio-conjugation. The surface of the particles can also be modified for attachment to an array by covalent or non-covalent bonds.

In order to prevent the entrapped particles in the aqueous phase from clogging up the pores of the membrane during emulsification, the pores 110 of the membrane 100 can be straight through pores 114. Membranes with straight through pores are available, for example, from Whatman (UK), PALL Corporation (USA), Micropore Technologies (UK) and Nanomi Emulsification Systems (NL). The above list is not intended to be exhaustive and membranes with straight through pores are also available from other suppliers. Some or all of the pores of a porous partition can be straight through pores. In addition, the membrane can be supported or reinforced (e.g., by a screen or housing). The pores can be sized such that the particles do not become entrapped during emulsification. Membrane emulsification using a membrane with straight through pores can be used to create droplets of a desired size and substantially uniform distribution.

The device can comprise a membrane which forms the partition 100. The porous partition 100 is supported by a partition holder ledge 120 within the partition holder 122. The partition 100 has at least two straight through pores 114 with a defined pore opening 110 having a shape. The partition 100 includes a first major surface 102 facing the aqueous phase 32 and a second major surface 104 facing the oil phase 42.

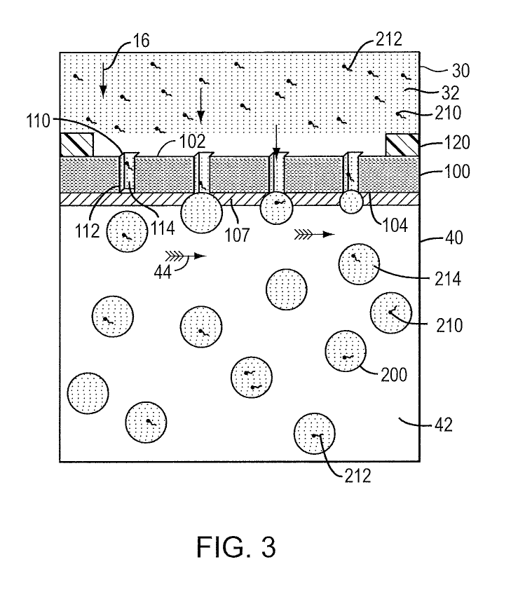

FIG. 3 shows an example of an expanded view of the partition 100 within the device. The partition 100 includes a first major surface 102 which can be hydrophilic and a second major surface 104 which can be hydrophobic wherein the pore interior wall surface 112 is independently hydrophobic or hydrophilic. The partition 100 acts to separate a first disperse aqueous phase 32 which is hydrophilic from a second continuous phase 42 which is hydrophobic. When the first phase 32 passes from the hydrophilic surface 102, through the straight through pore 114 and into the hydrophobic phase 42 a substantially uniform-size inverse emulsion droplet 200 is formed. Conversely, one of skill in the art is aware that when the hydrophobic phase 42 passes from the hydrophobic surface 104, through the straight through pore 114 and into the hydrophilic phase 32, a substantially uniform-size normal emulsion droplet is formed.

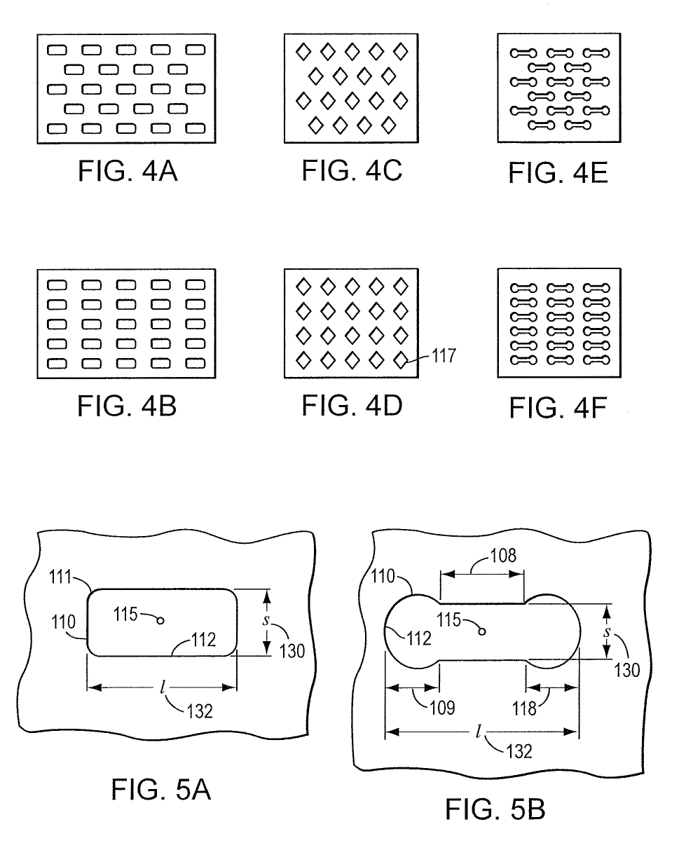

FIG. 4 illustrates both exemplary pore shapes in cross section and exemplary arrangements of the pores within the membrane 100. The shape of the pore 110 and that of it as seen in cross section can be a polygon, an oval, an oblong, a dumbbell, a bowtie, a kite and irregular shapes thereof. The polygon can be in the shape of a quadrilateral, a pentagon, a hexagon, a heptagon, an octagon, a nonagon, a decagon, and irregular shapes thereof and the quadrilateral can be in the shape of a kite, a rhombus, a trapezium, a trapezoid, an isosceles trapezoid, a parallelogram, a rectangle, and irregular shapes thereof. The polygon and bowtie shapes can have radius corners 111, FIG. 5A. The shape of the pore 110 is such that it has a defined length 132 of at least 4 microns to at least 16 microns and a defined width 130 of at least 1 micron to at least 4 microns, FIG. 5B.

The porous partition 100 can be a porous membrane with a controlled porosity ranging from 2 to 98% and a controlled pore size ranging from 1.0 to 200 μm. The number of pores per area can have numerous configurations. Example arrangements of pore density are 91,500 or 183,000 pores per 43.89 mm², 15,200 or 30,400 pores per 4 mm², 4200 pores per 2.1 mm², and 7000 pores per 1.8 mm². The number of pores per a given area is dependent upon the distance between pores (pitch) and the distance between rows of pores. The pitch distance between pores can be from 20, 21, 22, 23, 24, 25, 26, 27, 28, 29, and 30 μm and intervals therein and the distance between rows of pores can be 10, 15, 20, 25, or 30 μm and intervals therein. The thickness of the membrane can be 25, 30, 45 or 50 μm and intervals therein while the thickness of the non-porous area of the partition is between 400-500 μm. Example pore dimensions range in width from 1-4 μm, 4-16 μm in length and intervals therein. Example pores as measured in cross section (width×length) include 1.5×7.3 μm, 1.7×7.5 μm, 2.5×8.6 μm and so on. The porous partition 100 can be re-enforced between sections of the porous membrane or within the porous membrane using ridges or a screen, for example.

FIG. 5A and FIG. 5B illustrate the lengths and widths of both a rectangular pore with radiused corners 111 and a dumbbell shaped pore with a first 109 and second 118 lobe shaped pores. The pore 110 has an aspect ratio which is a measurement of the ratio of the length 132 of the pore 110 verses the width 130 of the pore. This can be depicted as:

l/s

where “l” is the length of the pore and “s” is the width of the pore.

The aspect ratio of the pore is at least 3:1, at least 3.1:1, at least 3.2:1, at least 3.3:1, at least 3.4:1, at least 3.5:1, at least 3.6:11, at least 3.7:1, at least 3.8:1, at least 3.9:1, at least 4:1, at least 4.1:1, at least 4.2:1, at least 4.3:1, at least 4.4:1, at least 4.5:1, at least 4.6:1, at least 4.7:1, at least 4.8:1, at least 4.9:1, at least 5.0:1, at least 5.1:1, at least 5.2:1, at least 5.3:1, at least 5.4:1, at least 5.5:1, at least 5.6:1, at least 5.7:1, at least 5.8:1, at least 5.9:1, at least 6.0:1, at least 6.1:1, at least 6.2:1, at least 6.3:1, at least 6.4:1, at least 6.5:1, at least 6.7:1, at least 6.8:1, at least 6.9:1, at least 7.0:1 and intervals within these ranges. The width 130 of the pore 110 is at least 1.0 micrometer and can be up to 200 micrometers. Possible l×s values are 6 microns by 1.8 microns for an aspect ratio of 3.3:1, 7.9 microns by 1.8 microns for an aspect ration of 4.4:1 and 9.5 microns by 2.7 microns for an aspect ratio of 3.5:1.

The straight through pore 114 can be designed such that a particle having a size ranging from 0.1 microns to 100 microns can passing through the membrane without being caught in the straight through pore 114 and so block the flow of the first phase into the second phase. The particle 210 can have a size from 1.0 to 4.0 microns and intervals therein and the pores of the partition 100 can have a pore 110 size from 1.0 to 200 microns and intervals therein. The straight through pore 114 can be described as having a longitudinal axis 115 traversing the partition 100. FIG. 5B illustrates the structure of a dumbbell shaped lobe shaped pore in cross section. The lobe shaped pore has a central portion 108 as the axis of the lobe shaped pore and at least one lobe shape 109 contiguous with the central portion 108 and extending radially from the longitudinal axis 115. The lobe shaped pore can run parallel to the central longitudinal axis 115 of the straight through pore 114 as well as comprise a second lobe 118 being symmetrically positioned with respect to the first lobe shape 109, extending radially from the longitudinal axis 115 and being separated from the first lobe 109 by the central portion 108. The aspect ratio of bilaterally symmetrical lobe shaped pores is determined from the length 132 between the first and second lobes greater than a width 130 of the central portion 108 separating the first and second lobes, FIG. 5B. The width (s) 130 of the central portion 108 is a measurement made perpendicular to the length 132 of the central portion 108 and the length (l) 132 is a measurement made perpendicular to the width (s) 130 of the central portion 108 and spanning the distance of an imaginary line drawn between the opposite ends of the length of the pore as viewed in cross section. Exemplary examples of loped shaped pores are bowtie and dumbbell shapes. Loped shaped pores can be bilaterally symmetrical or nonsymmetrical. Lobe shaped pores can include pores with at least one lobe.

The first phase 32, a hydrophilic phase, can further comprise a particle 210. The particle can have a shape selected from a bead, a disc, a cube, a pyramid, a sphere, a polyhedron and irregular shapes thereof. The particle 210 can also be magnetic or have attached to it a biotin/streptavidin like moiety and further comprise an oligonucleotide 212 attached to the particle. The aqueous phase 32 can further comprise reagents for performing a polymerase chain reaction (PCR). The PCR reaction can occur within the discrete phase 214 within the inverse emulsion of the monodispersed, substantially uniform-size emulsion droplet 200. The PCR reagents can include buffer, a salt such as magnesium chloride, at least one primer, dNTPs, a template, a polymerase and other reagents for amplification of the oligonucleotide 212 attached to the particle 210 within the emulsion droplet 200.

The membrane can be made of a polymer. Exemplary polymers include, but are not limited to, poly(ether sulfone), polyester, polycarbonate, polyimide, polytetrafluoroethylene (PTFE), and other fluorinated and perfluorinated polymers. The surfaces of the polymer membranes can be roughened (e.g., to nanometer scale). Surface roughening can be accomplished mechanically or chemically. For example, a surface of the porous partition can be roughened by oxygen plasma in order to achieve super-hydrophobicity. Other methods of rendering the surface of the porous partition hydrophobic can also be employed. Methods of this type are disclosed in Li et al., Chem. Soc. Rev., 2007, 36(8), 1350-1368.

The membrane can also be made of glass such as, for example but not limited to, soda lime glass or Shirasu glass. The membrane can also be a thin metal foil of, for example, stainless steel or another metal alloy. The membrane can also be made of silicon. The membrane can also be a silica or alumina membrane (e.g., made by sintering silica or alumina powders) or a porous ceramic membrane. The surface of the porous partition can be physically or chemically modified to tailor its hydrophilicity.

The porous (track-etched) silicon membrane can be fabricated using photo-lithography, chemical etching, and reactive ion etching, RIE). Such methods are well known to one of ordinary skill in the art. Using a photomask with 1.5 to 10 μm holes, the underlying gold layer is chemically etched. In a subsequent step, holes are drill through the bulk of the membrane by reaction ion etching.

In another embodiment, the surfaces 102 and 104 of the partition 100 can both be hydrophobic or hydrophilic or independently hydrophobic or hydrophilic. For example, pores are formed in the partition using photolithographic techniques to lay down a photo mask atop the silicon membrane surface 102 or 104 and chemically treating the silicon membrane to form the straight through pores 114 and step-wise silylation of each surface to impart separate hydrophilic and hydrophobic surfaces 102 and 104 or mutually hydrophilic or hydrophobic surfaces 102 and 104. The interior wall 112 of the straight through pore 114 can have its surface 113 modified in conjunction with the unmasked surface.

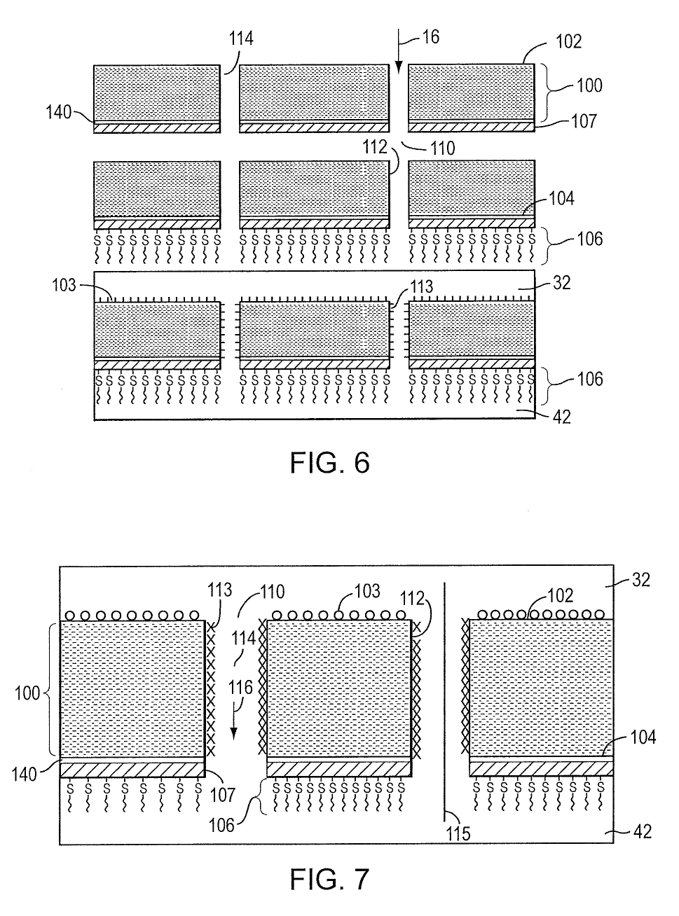

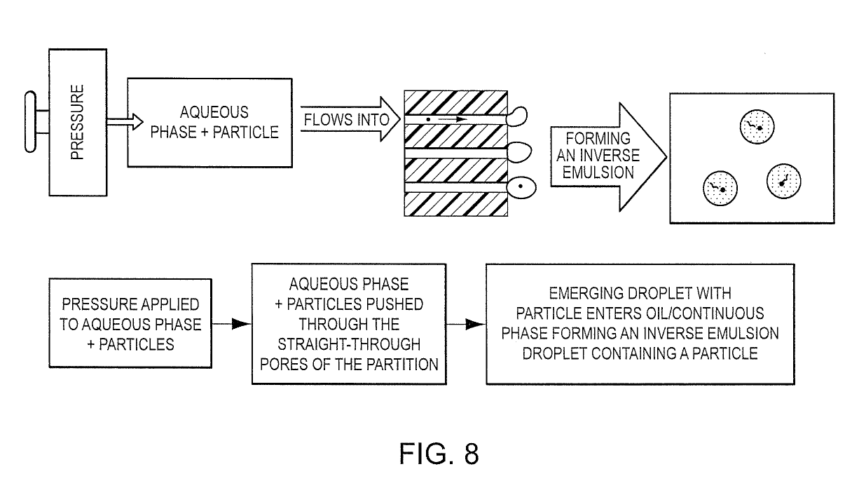

FIG. 6 illustrates possible surface treatments of the porous partition 100 for forming inverse emulsions. The first major surface 102 of the partition can be chemically modified to render both the surface 102 and the interior wall surface 112 hydrophilic. The second major surface 104 can be covered entirely by a mask (including the pore openings 110) to make a barrier for the chemical treatments to surfaces 102 and 112. Such treatments include chemically bonding a poly(ethylene oxide) (PEO) or poly(ethylene glycol) (PEG) moiety (a process hereinafter referred to as “PEGylating”) to surfaces, such as silicon and silicon dioxide. The second major surface 104, facing the oil phase can be treated by to make it receptive to chemical treatments to render the surface 104 hydrophobic.

As shown in FIG. 7 the second major surface 104, can be prepared for chemical surface modifications by applying a layer of an adhesion enhancer 140, for example but not limited to, chromium, in preparation of applying a layer of a coinage metal 107, for example, gold or other materials known to one of skill in the art. Examples of coinage metals include, but are not limited to copper, nickel, gold, platinum, bronze and zinc. This exemplary list is not intended to be limiting and other metals known to one of skill in the art can also be used. The bare gold surface 107 can be subjected to thiolation using an alkyl thiol of a perfluoroalkyl thiol, rendering its surface hydrophobic 106. Those with ordinary skills in the art can appreciate other sulfur containing compounds, for example, but not limited to, dialkyldisulfides and its fluorinated versions, and other oligomeric and polymeric compounds comprising thiol and/or disulfide groups can be used to render the gold surface hydrophobic. Various types of linear alky and branched alky thiols can be synthesize according to U.S. Pat. No. 5,395,550 (1995). Perfluorinated thiol compounds can be synthesized according to the procedures reported by (C. S. Rondestvedt, et al., J. Org. Chem. 42:2680-2683 (1977)). These references are incorporated by reference herein in their entirety. Typical examples for thiolation of gold are:

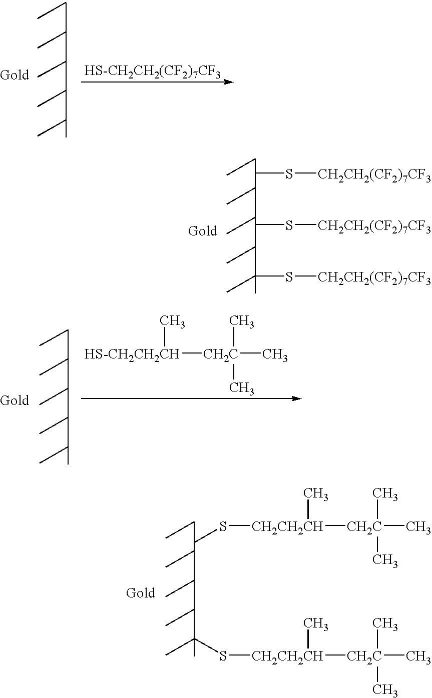

After thiolation to render the gold surface 107 hydrophobic 106, the remaining surfaces 102, including the inner wall surfaces 112 inside the pores 114, can be PEGylated to become hydrophilic 113.

Surface PEGylation of surfaces 102 and 112 can be implemented with a mono-, di-, or tri-alkoxysilane comprising a co-methoxy-poly(ethylene oxide), MeO-PEO, or poly(ethylene glycol), PEG moiety comprising from about 5 to about 10000 repeating units, for example, from about 6 to about 300 repeating units, or, for example, from about 10 to about 200 repeating units. Those skilled in the art would be able to determine the number of repeating units of the PEO or PEG moiety to achieve desired surface features.

Various reactions can be effected to PEGylate a surface in accordance with the disclosure. One of skill in the art will appreciate that PEGylation can be achieved

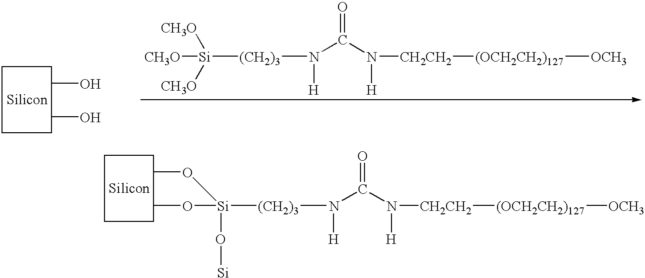

by many other reactions which, although not specifically discussed herein, are within the scope of the invention. The following reaction is an example of surface PEGylation on a silicon substrate using a trimethoxysilane having a MeO-PEO moiety.

FIG. 7 illustrates an example of the surface treatment of the partition 100 surface to facilitate formation of substantially uniform-size inverse emulsion droplets. The first major surface 102 and interior walls 112 of the straight through pores 114 undergo PEGylation to render the first major surface hydrophilic 103 as well as the interior wall surfaces 113 of the straight through pores 114. Exemplary PEGylation protocols are taught in US 20060091015A1 (2006) and US 2007009566A1 (2007)), each reference is incorporated by reference herein in their entirety. One of skill in the art will appreciate that the gold surface 107 can be rendered hydrophilic with a PEG or PEO comprising thiol or disulfide groups, and the remaining surfaces 102 and 112 can be rendered hydrophobic with an alkyl or perfluoroalkyl alkoxysilane, resulting in a membrane for the preparation of normal emulsions.

As shown in FIG. 1 and FIG. 2, when an external pressure 16 is applied to the aqueous phase 32 in which particles 210 are suspended, the aqueous phase 32 is forced through the straight through pore 114 channels of the membrane 100 into the continuous phase 42. The continuous phase 42 may be flowing 44 (e.g., swirling) to sweep across the membrane 100 surface 104 thereby carrying droplet 200 with or without a particle 210 encapsulated to reservoir 60. For a given membrane with pre-determined pore size, the droplet size can be controlled by a set of parameters including, but not limited to, the applied pressure to the aqueous phase, the viscosity of the aqueous phase, the viscosity of the continuous oil phase, the shear force generated by the flow of a continuous phase, the aspect ratio of pore 110, and the nature and the concentration of the surfactant(s) used.

Additionally, the hydrophilicity and hydrophobicity of the surfaces of the porous membrane 100 can also affect droplet; size, formation, rate of formation and uniformity. It is therefore desirable the surface of the membrane in contact with the continuous oil phase is hydrophobic while the interior wall surfaces are hydrophilic in order to lower the applied pressure for the preparation of inverse emulsion,

Pc=4γ cos θ/ d̅p

where Pc is the critical pressure, γ the oil/water interfacial tension, θ the contact angle of the oil droplet against the membrane surface well wetted with the continuous phase and d̅p the average pore diameter. For example, a hydrophilic surface 103 facing the aqueous phase as well as a hydrophilic interior wall surface 113 will facilitate passage of the aqueous phase 32 through the porous membrane 100 with less applied pressure when other variables such as viscosity and surfactant(s) used are consistent verses a porous membrane with all hydrophobic surfaces.

Each discrete droplet 200 comprising reagents in its dispersed aqueous solution 214 and having a particle 210 entrapped therein can be used to conduct reactions such as polymerase chain reactions (e.g., emulsion PCR) or ligation reactions. Emulsion PCR protocols are described in Williams et al., Nature Methods 3(7):545-550 (2006); Diehl et al., Nature Methods 3(7):551-559 (2006); and Miller et al., Nature Methods 3(7):561-570 (2006).

The emulsification apparatus and methods described herein can be used to provide water-in-oil emulsions having substantially uniform droplet size. Modifications to the interior surface of the straight through pores to render them hydrophilic results in lower pressure needed to force the aqueous phase through the membrane. Additionally, the majority of the pores are operational and functioning to facilitate the passage of the aqueous phase into the oil phase and thus, droplet formation. This will result in improved rates of emulsion formation, more uniformly sized droplets being produce and improved droplet size distribution. The modified pore shapes as viewed in cross section with aspect ratios greater than 4 to 1 also facilitate ease of formation of uniformly sized, reproducible and enhanced rate of inverse emulsion droplet formation.

In an application where a particle is entrapped in a droplet and a subsequent reaction (e.g., PCR) is conducted within the droplet, substantially uniform droplet size can improve the reliability of the results. For example, the uniform droplet size can improve the likelihood that all particles will be surrounded by an environment that contains approximately the same total reactant content, thereby providing more uniform reaction results in the droplets. In addition, the substantially uniform-size of all droplets can result in the emulsion generating system following the Poisson Distribution model for droplets with a single particle. Droplets of sizes which vary over a wide distribution range will cause the emulsion generating system to depart from the Poisson model in complex and unpredictable ways, adversely affecting predictability of results other than by empirical methods, which will vary as the distribution range varies.

The methods described herein can be used in a variety of potential applications, including in vitro evolution of proteins and RNA's (see, for example, U.S. Pat. No. 6,489,103 B1), cell-free cloning and sequencing. These techniques can be used in any application where a diverse collection of DNA or RNA fragments are amplified or modified in isolation from each other using a set of amplification or modification reagents.

Those having ordinary skill in the art will understand that many modifications, alternatives, and equivalents are possible. All such modifications, alternatives, and equivalents are intended to be encompassed herein.

### EXAMPLE 1

### Surface Modification of Porous Silicon Layer

As set forth above, the porous partition can be a porous silicon layer. A surface of the porous silicon partition can be modified to render it hydrophobic. The porous silicon partition having a hydrophobic surface can be used in an apparatus as described herein with the hydrophobic surface facing the continuous oil phase.

The surfaces of the porous silicon layer (membrane) can contain a surface layer of native oxide or oxide grown by chemical means. This oxide surface layer can be modified with alkyl- and/or fluorinated alkylsilanes (1), resulting in a surface having hydrophobic characteristics.

R₁—Si(R₂)ₓ(R₃)_{y} (1)

Where:

R₁=CₙH₂₊₁ or CH₂CH₂(CF₂)ₘCF₃;

R₂=C_{q}H_{2q+1};

R₃=OR₂ or Cl;

X=0 to 2;

x+y=3;

n and m are independently integers of 4 to 25; and

q=1 to 5.

After silylation, the surface fluorinated alkyl groups render the surface hydrophobic. These fluorinated alkyl groups can also act as a tie-layer to enhance the adhesion of an additional top coating of perfluorinated polymer such as TEFLON® AF or CYTOP, the structures of which are set forth below.

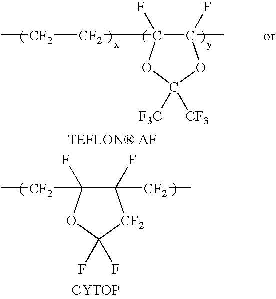

These two perfluorinated polymers are soluble in perfluorinated hydrocarbon solvents and the solutions can be spin-casted, dip-coated, or spayed onto surfaces to improve surface hydrophobicity. Super hydrophobic surfaces can be achieved by coating a monolayer of these polymers onto surfaces with roughness in nanometers scale (Li et al., Chem. Soc. Rev. 36(8):1350-1368 (2007)).

The following procedures are representative of procedures that can be employed for the surface modification of silicon wafers/membranes.

### EXAMPLE 2

### Procedure for Pre-Treatment Prior To Surface Chemical Modification

A silicon wafer with a mirror surface or a porous silicon membrane, for example, 17 mm×17 mm, can be sonicated in 30 ml of 1.0% sodium dodecylsulfate (SDS) for 20-60 minutes. The wafer/membrane can then be thoroughly rinsed with deionized water. The wafer/membrane can be subsequently sonicated in a mixture of 5 mL of 29% NH₄OH, 5 ml of 30% H₂O₂, and 20 mL of DI water for 20-60 minutes. It can then be rinsed with DI water thoroughly. The silicon wafer/membrane can then be sonicated in a mixture of 5 mL of 38% HCl. 5 mL of 30% H₂O₂, and 20 mL of DI water for 20-60 minutes and rinsed with DI water thoroughly. The silicon wafer/membrane can then be dried (e.g., blow-dried) with nitrogen and used immediately. A typical static water contact angle (2 μL of water deposited by a micropipette manually) for a pretreated wafer/membrane is ≦10 degrees.

### EXAMPLE 3

### Procedure for Surface Chemical Modification Using Alkylsilane Reagents

Into 35 ml of 100% EtOH, 1.0 mL of decyltriethoxysilane can be added and stirred to dissolve. The pre-treated silicon wafer or porous silicon membrane can then be soaked in this silane solution for 30 minutes while agitated (e.g., with an orbit shaker). The wafer/membrane can then be removed and dipped into 100% ethanol briefly and excess solvent is shaken off. The wafer/membrane can then be cured at 110° C. for 20 minutes. A typical static water contact angle (2 μL of water deposited by a micropipette manually) for an alkylsilylated chip is about 90 degrees.

### EXAMPLE 4

### Procedure for Surface Modification Using Fluorinate Silanes

Into a 15 ml glass vial with a screw cap, 4 mL of a fluorinated solvent, for example perfluoro-(2-perfluoro-n-butyl)tetrahydrofuran (FC-75 obtained form 3M) and 0.5 mL of a fluorosilylating reagent, for example (hepetadecafluoro-1,1,2,2-tetrahydrodecyl)trimethoxysilane or (hepetadecafluoro-1,1,2,2-tetrahydrodecyl)dimethylchlorosilane (obtained from Gelest) can be mixed to dissolve. The pre-treated and blow-dried silicon wafer with a mirror surface or porous silicon membrane can be soaked in this silane solution for 20 minutes with occasional agitation. The wafer/membrane can then be removed and the excess silane solution is removed by shaking and dried (e.g., blow-dried) with nitrogen. The chip can then be cured at 110° C. for 20 minutes. A typical static water contact angle (2 μL of water deposited by a micropipette manually) for a fluorosilylated silicon wafer/membrane is about 110 degrees.

### EXAMPLE 5

### Procedure for Coating a Fluorosilylated Silicon Chip with a Perfluorinated Polymer

A 0.5% w/v solution of a perfluorinated polymer, for example, TEFLON® AF-1600 (obtained from DuPont) or CYTOP (obtained from Asahi Glass) can be prepared using a fluorinated solvent, for example, FC-75 (obtained from 3M) or CTSOLV-180 (obtained from Asahi Glass). The fluorosilylated silicon wafer having a mirror surface or porous silicon membrane can be dipped into the solution briefly, excess of solution shaken off and the coated wafer/membrane can be cured at 110° C. for 20 minutes. A typical static water contact angle (2 μL of water deposited by a micropipette manually) for a coated wafer/membrane is about 120 degrees.

### EXAMPLE 6

### General Procedure for Solution PEGylation to Render a Surface of a Porous Silicon Membrane Hydrophilic

Typically, the porous silicon membrane is pretreated prior to PEGylation. After the removal of the photo resist by sonicating the membrane in an organic solvent, for example, PRS-3000™ Positive Photo resist Stripper, obtained from J. T. Baker, the surfaces are rinsed thoroughly with plenty of ethanol, blow-dried with a stream of nitrogen, and then baked in a convection oven at 110° C. for 30 minutes. The cleaned silicon membranes are then treated according to Example 2. The pretreated silicon membranes were used immediately for PEGylation.

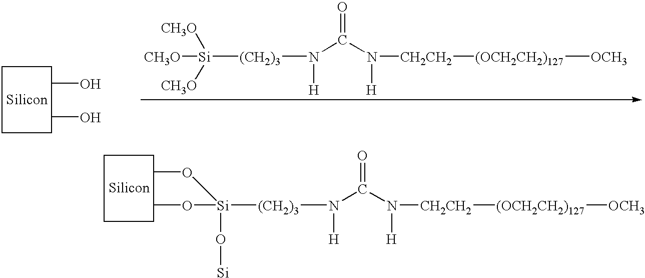

A TEFLON™ box constructed in such a way that it had a cavity of 35 mL and slots at the bottom to hold 10 porous silicon membranes, 17 mm×17 mm in size, at vertical position, was used for solution PEGylation. The dimensions and capacity of this TEFLON™ box can be scaled up to hold more membranes. After placing the membranes in the TEFLON™ PEGylation box, the airtight cap containing an inlet and an outlet capped with rubber septums, was replaced and sealed. The PEGylation box was then purged with ultra-pure argon at 500 mL per minute for 2 minutes. A solution of 0.5 mL of 2-[methoxy(polyethyleneoxy)propyl]trimethoxysilane, MW of 5,910 Da, (Nektar, Huntsville, Ala.) in 30 mL of anhydrous tetrahydrofuran (THF) was added using a syringe. It was followed by adding 1.0 mL of triethylamine. The sealed PEGylation box was rocked on a Cole-Parmer Rocking Platform at 55 rpm. After 20 hours of rocking the membranes were removed, rinsed briefly with THF, blow-dried with a stream of nitrogen, and baked in a convection oven at 110° C. for 10 minutes. The PEGylated membranes were kept in a covered Petri dish under ambient conditions prior to use. Static water contact angle was measured using Drop Shade Analysis System DSA100 obtained from Kruss, Matthews, N.C. A total of 12 data points were taken from three random samples. The average static water contact angle was 32.1 degrees with a standard deviation of 0.5 degrees.

### EXAMPLE 7

### Mercaptosilylation of Silicon Membrane Prior to Vapor Deposition of Gold

The porous silicon membrane is prepared by wiping and then rinsing the membrane with analytical grade acetone using a wash bottle. The membrane is then sonicated in Milli-Q water with 0.5 wt % of Triton X-100, 15 min. and then rinsed with copious amounts of Milli-Q water and then blown dry with nitrogen prior to use.

Silylation of the membrane is done using the method of A. Ross et al., J. Mater. Chem., 13:722-726 (2003) with modifications. Prepare a fresh solution of 90.1 μL 3-mercaptopropyl(methyl)dimethoxysilane, 6.6 μL hexylamine and 100 mL toluene and mix under nitrogen. The membrane is soaked in the solution for 15 min. The membrane is removed from the solution and rinsed with HPLC grade toluene and blown dry with a stream of N₂. The silylated membrane is used immediately for vapor deposition of gold.

### EXAMPLE 8

General Procedure for Thioalkylation to Render a Gold Surface Hydrophobic

The surface of the porous silicon membrane is prepared for chemical surface modifications, including adding a gold layer by applying an adhesion enhancer such as chromium. The gold can be deposited by a vapor deposition process and is then subjected to thiolation using an alkyl thiol or a perfluoroalyl thiol to render the surface hydrophobic.

### EXAMPLE 9

### General Procedure o Render a Gold Surface Hydrophilic

The gold surface is initially cleaned with a Piranah solution to enhance surface density of silanol groups with OH groups bonded to the silicon substrate. A silylation process follows to treat the surface of the porous silicon membrane on which the gold layer is to be deposited. The mercapto-containing silylating agent (obtained from Gelest, Inc.) reacts with the OH groups on the surface of the membrane, resulting in the incorporation of surface hydrogen sulfide groups (HS). The silyation process ultimately provides good adhesion of the gold to the porous silicon membrane because the formed HS groups (e.g., mercapto groups) react with and form chemical bonds with gold. After the silylation process, the gold is deposited on the substrate via a vapor deposition process. The mercapto functional groups also resulted in strong adhesion of the transparent gold layer to the membrane surface, as covalent bonds are formed between the deposited gold and the sulfur (S).

The deposited gold layer is then subject to a PEGylation process to render the gold surface hydrophilic. The gold surface is exposed to an aqueous tetrahydrofuran (THF) solution containing a mercapto-functionalized poly(ethylene glycol) (molecular weight 5,723 Da, obtained from Nektar). The mercapto groups form a strong covalent bond with the gold layer via the sulfur (S) bond. The resulting gold surface layer has poly(ethylene glycol) groups (PEG) bonded to the gold.

### EXAMPLE 10

### Preparation of Straight-Through Holes within the Partition

A silicon wafer or silicon partition can be fabricated to include straight through pores (porous membrane) using common chemical practices known to one of skill in the art. For example, beginning with a silicon on insulator (SOI) wafer having a top silicon layer between 30-50 microns in thickness which lies atop a 2 micron oxide layer atop 350-450 micron layer of silicon a photoresist (PR) mask is spun onto the top surface. Photolithography is performed to pattern the PR mask for the pore shapes, density and layout. Dry reactive ion etching (DRIE) is done to the top layer to etch the top 30-50 micron silicon layer down to the oxide layer wherever the PR does not cover the silicon. The PR mask is then removed and a second PR mask is applied to the backside of the SOI wafer (the 350-450 micron thick surface) and photolithography is performed to pattern the PR mask for the membrane (i.e., porous region) of the partition. DRIE is used to etch the 350-450 micron layer of silicon up to the oxide layer followed by removal of the mask. The silicon wafer or silicon partition is then dipped into hydrofluoric acid to remove the oxide layer opening the pores in the top silicon layer into the thick silicon layer forming the porous membrane area of the partition.

### EXAMPLE 11

### Preparation of Continuous Phase and Aqueous Phase

The continuous phase can include 4-10 wt % and 0.2-1.5 wt % of SPAN-80 and TWEEN-80, respectively, in mineral oil or other suitable solvents. The following table illustrates the composition of an aqueous phase containing from 1 to 2 billion beads, for example 1.6 to 1.7 billion PI beads for use in a PCR reaction. P1 and P2 designate primers. The aqueous phase can be scaled up as needed.

| | | |
|---|---|---|
| GeneAmp ® 10X PCR Gold Buffer | 280 | μL |
| 100 mM dNTP | 98 | μL |
| 1.0 M MgCl | 70 | μL |
| 10 μM P1-soln | 11.2 | μL |
| 500 μM P2-soln | 16.8 | μL |
| Template | 0.2 | pg |
| Nuclease free water | 1560 | μL |
| 5 U/μL Ampli Taq Gold ® DNA Polymerase, UP | 594 | μL |

Those who are skilled in the art will appreciate that the above-mentioned procedures can be applied to silicon wafers/membrane or chips whose surfaces comprise artificial features which include, but are not limited to, holes, straight-through-hole, posts, pillars, spikes, grooves, pits, indentations or fractal structures. The surface of a silicon wafer/mask can also be roughened mechanically or chemically to have a surface roughness of nanometer to micrometer scale.

While the foregoing specification teaches the principles of the present invention, with examples provided for the purpose of illustration, it will be appreciated by one skilled in the art from reading this disclosure that various changes in form and detail can be made without departing from the spirit and scope of the invention.

What is claimed is:

1. A device for forming a plurality of substantially uniform-size emulsion droplets, at least one emulsion droplet comprising a particle, comprising:
a.) a first chamber containing an aqueous phase comprising a plurality of particles;
b.) a second chamber containing an oil phase which comprises a water-immiscible liquid; and
c.) a partition separating the first chamber from the second chamber, said partition comprising at least a first major surface, a second major surface opposite the first major surface, and at least two defined straight through pores connecting the first major surface to the second major surface, where said first major surface forms a wall of the first chamber and said second major surface forms a wall of the second chamber;
wherein when said aqueous phase passes through two of said at least two straight through pores to said oil phase said aqueous phase forms a plurality of discrete substantially uniform-size emulsion droplets, and at least one of the plurality of droplets comprises at least one particle of the plurality of particles.

2. The device of claim 1, wherein said defined pore has a cross sectional shape.

3. The device of claim 2, wherein said cross sectional shape comprises a polygon, an oval, an oblong, a dumbbell, a bowtie, a kite and irregular shapes thereof.

4. The device of claim 2, wherein said cross sectional shape comprises a minimum pore dimension of at least 1.0 to 4.0 micrometers.

5. The device of claim 3, wherein said cross sectional shaped pore has an aspect ratio of at least 4 to 1.

6. The devise of claim 1, wherein at least one of said first major surface and said second major surface, and two of said at least two straight through pores comprise at least one hydrophobic surface or at least one hydrophilic surface.

7. A partition through which to pass a first phase into a second phase to form substantially uniform-size emulsion droplets, comprising:
a.) a first major surface;
b.) a second major surface opposite the first major surface; and
c.) at least two straight through pores, each of said at least two straight through pores comprising a cross sectional shape selected from the group consisting of a polygon, an oval, an oblong, a dumbbell, a bowtie and irregular shapes thereof and an interior wall traversing the partition between the first major surface and the second major surface, and
wherein said partition is adapted to form substantially uniform-size emulsion droplets comprising the first phase in the second phase.

8. The partition of claim 7, wherein said cross sectional shape comprises a defined length (l) of from about 4 to 16 microns and a defined width (s) of from about 1 to 4 microns.

9. The partition of claim 8, wherein said cross sectional shape comprises an aspect ratio, length to width, of at least 4 to 1.

10. The partition of claim 7, wherein said polygon is selected from the group consisting of a quadrilateral, a pentagon, a hexagon, a heptagon, an octagon, a nonagon, a decagon, and irregular shapes thereof.

11. The partition of claim 10, wherein said quadrilateral is selected from the group consisting of a kite, a rhombus, a trapezium, a trapezoid, an isosceles trapezoid, a parallelogram, a rectangle, and irregular shapes thereof.

12. The partition of claim 7, wherein said cross sectional shape is parallel to a central longitudinal axis in the straight through pore.

13. The partition of claim 12, wherein said cross sectional shape is bilaterally symmetrical to said central longitudinal axis.

14. The partition of claim 7, wherein said first phase comprises a plurality of particles comprising at least one nucleic acid attached thereto.

15. A method of forming substantially uniform-size emulsion droplets comprising:
forcing an aqueous phase comprising a plurality of particles in contact with the first major surface of a partition through at least two straight through pores in the partition and into a dispersion medium, and
simultaneously,
moving the dispersion medium parallel to and in contact with a second major surface of the partition wherein the second major surface is opposite the first major surface, thereby forming substantially uniform-size emulsion droplets of the aqueous phase dispersed in the dispersion medium, and
wherein a plurality of the droplets comprise at least one particle.

16. The method of claim 15, wherein said straight through pore comprises an interior wall, a portion of said interior wall being designed for forming said substantially uniform-size emulsion droplet.

17. The method of claim 16, wherein said straight through pore comprises a cross sectional shape.

18. The method of claim 17, wherein said cross sectional shape comprises a defined length (l) of from about 4 to 16 microns and a defined width (s) of from about 1 to 4 microns.

19. The method of claim 18, wherein said cross sectional shape comprises an aspect ratio, length to width, of at least 4 to 1.

20. The method of claim 17, wherein said cross sectional shape comprises a shape selected from the group consisting of a polygon, a circle, an oval, an oblong, a dumbbell, a bowtie and irregular shapes thereof.

21. The method of claim 15, wherein attached to each of at least some of said plurality of particles is at least one nucleic acid.

22. The method of claim 21, wherein said aqueous phase further comprises reagents for performing a polymerase chain reaction (PCR).

23. The method of claim 22, wherein said polymerase chain reaction occurs within said substantially uniform-size emulsion droplet.

24. The method of claim 23, wherein said nucleic acid is amplified by said polymerase chain reaction.
